(19) United States
(12) Patent Application Publication
(10) Pub. No.: US 2005/0171052 A1
(43) Pub. Date: Aug. 4, 2005

(54) Sucrose acetate isobutyrate formulation

(76) Inventors: Phillip Michael Cook, Kingsport, TN (US); George Chester Zima, Kingsport, TN (US); Danessa Leann Sexton, Johnson City, TN (US)

Correspondence Address: Jonathan D. Wood;Eastman Chemical Company, P.O. Box 511, Kingsport, TN, 37662-5075, US

(21) Appl. No.: 10/989,064

(22) Filed: Nov. 15, 2004

Related U.S. Application Data
(60) Provisional application No. 60/520,066, filed on Nov. 14, 2003.

(51) Int. Cl. A61K 31/7024

(52) U.S. Cl. 514/53

(57) ABSTRACT

The present invention provides a solid sucrose acetate isobutyrate formulation having improved handling characteristics.

This application claims the benefit of U.S. Provisional Application Ser. No. 60/520,066, filed Nov. 14, 2003.

### FIELD OF INVENTION

This invention relates to sucrose acetate isobutyrate (SAIB) formulations, specifically SAIB formulations having improved handling characteristics.

### BACKGROUND OF THE INVENTION

Sucrose acetate isobutyrate ((SAIB) is an additive for use in beverages, cosmetics, pharmaceuticals and other applications. While SAIB has many exceptional benefits in these and other applications, its high viscosity presents practical difficulties in handling. For example, at room temperature SAIB is a sticky material having a viscosity of greater than 100,000 cP, making pouring practically impossible.

To overcome these handling problems, SAIB can be heated or diluted to decrease its viscosity, allowing SAIB to be handled as a liquid. In beverage applications, food grade solvents are used as diluents to make SAIB less viscous (approximately 1,000 to 10,000 cP) and hence more pourable. Eastman Chemical Company, Kingsport, Tenn., currently markets three low-viscosity products: Sustane SAIB-FG CO (containing 10% orange terpenes), Sustane SAIB-FG ET-10 (containing 10% ethanol), and Sustane SAIB MCT (containing 20% medium chain triglycerides). While resolving the viscosity issues associated with SAIB, the low viscosity SAIB may have certain characteristics that can make them less than desirable in certain applications. For instance, certain solvents, or other auxiliary ingredients may be either undesirable in some formulations or not approved for use in certain countries. Ethanol, for example, is not allowed for use by some cultures.

In view of these limitations, there is a need for an SAIB formulation having improved handling characteristics.

### BRIEF SUMMARY OF THE INVENTION

The present invention overcomes the above mentioned handling problems by providing an SAIB formulation comprising sucrose acetate isobutyrate in an amount from about 1 weight percent to about 80 weight percent based on the total weight percent of the total formulation; and a substrate, wherein the substrate is present in an amount from about 99 weight percent to about 30 weight percent based on the weight of the formulation, wherein the SAIB formulation is a solid and wherein the SAIB formulation is pourable in less than about 20 seconds according to ASTM method D1895-96.

### DETAILED DESCRIPTION

The present invention may be understood more readily by reference to the following detailed description of the invention and the examples provided therein. It is to be understood that this invention is not limited to the specific formulations, blends, processes and conditions described, as specific formulations, blends, processes and/or process conditions may, of course, vary.

It must also be noted that, as used in the specification and the appended claims, the singular forms “a,” “an” and “the” include plural referents unless the context clearly dictates otherwise.

Ranges may be expressed herein as from “about” a particular value and/or to “about” another particular value. When such a range is expressed, another embodiment includes from the one particular value and/or to the other particular value.

By “comprising” or “containing” is meant that at least the named compound, element, particle, or method step etc. must be present in the composition or article or method, but does not exclude the presence of other compounds, materials, particles, method steps, etc., even if the other such compounds, materials, particles, method steps etc. have the same function as what is named.

Unless otherwise specified, weight percent is based on the total weight of the formulation, blend or other combination. For example, as stated herein, a sucrose acetate isobutyrate (SAIB) formulation comprising from about 1 weight percent to about 80 weight percent sucrose acetate isobutyrate, and from about 30 weight percent to about 99 weight percent of a substrate. The weight percentages of the sucrose acetate isobutyrate and the substrate are based on the total weight of the SAIB formulation.

As used herein the term substrate refers to a material to which SAIB is combined to form a solid SAIB formulation that has improved handling characteristics, such as pourability as defined in ASTM D1895-96. In a preferred embodiment, the terms “pourable” and “pourability” may be used interchangeably to refer to the formulation being “pourable” as defined in ASTM D1895-96 in less than about 20 seconds.

As used herein, the term solid means a substance that is not a gas or a liquid at room temperature or temperature of use. The SAIB formulation in the present invention is in a solid form.

While the use of SAIB as an additive In various applications has many benefits, it is very difficult to handle. The present invention provides a solid SAIB formulation that has improved handling characteristics.

The present invention is directed to a solid SAIB formulation comprising SAIB and a substrate, wherein the formulation is pourable in less than about 20 seconds according to ASTM method D1895-96. In a preferred embodiment the solid formulation is pourable in less than about 15 seconds, more preferably less than about 10 seconds and most preferably pourable in less than about 5 seconds.

The SAIB is present in the formulation in an amount from about 1 weight percent to about 80 weight percent, preferably in an amount from about 30 weight percent to about 70 weight percent and more preferably in amount from about 40 weight percent to about 60 weight percent. In the most preferred embodiment, the SAIB is present in the formulation in an amount from about 40 weight percent to about 55 weight percent.

SAIB is commercially available from Eastman Chemical Company, Kingsport, Tenn., and can be prepared using known techniques by reacting sucrose with acetic and isobutyric anhydrides followed by extensive purification using high vacuum distillation. The degree of esterification is nearly complete (e.g., with the degree of substitution being greater than 7.5 and the maximum degree of substitution being 8), and the approximate ratio of acetate:isobutyrate esters is 2:6. U.S. Pat. No. 3,096,324 provides an example of the preparation of SAIB.

The substrate is any composition that can absorb or adsorb SAIB to form a solid formulation that is pourable in less than 20 seconds, including sucrose, hydrophobically modified food starch, gum acacia, maltodextrins including soluble maltodextrin fibers (commercially available from Matsutani, and known as Fibersol-2), cyclodextrins, microcrystalline cellulose, silica, titanium dioxide, carboxymethylcellulose, gum ghatti, modified gum ghatti, xanthan gum, tragacanth gum, guar gum, or other suitable gums, inorganic substrates such as sodium/potassium sulfate, talc, bentonite and various clays, waxes such as candellila, hydrocarbon and carnauba waxes. The preferred substrate will depend on the application in which the formulation is used. For example, if the end use of the SAIB formulation is in beverages, the preferred substrate is soluble in water or in oil such as sucrose, hydrophobically modified food starch, gum acacia, maltodextrins, including soluble maltodextrin fibers (commercially available from Matsutani America, Inc., and known as Fibersol-2), cyclodextrins, microcrystalline cellulose, carboxymethylcellulose, gum ghatti, modified gum ghatti, xanthan gum, tragacanth gum, guar gum, or other suitable gums, waxes such as candellila, hydrocarbon and carnauba waxes. The preferred substrate for cosmetic uses is clay, silica and titanium dioxide.

The substrate is present in an amount from about 30 weight percent to about 99 weight percent, is preferably present in an amount from about 40 to about 60 weight percent.

The weight ratio of SAIB to the substrate is dependent upon many factors, including the end use of the SAIB formulation, the manner of preparation, nature and porosity of the substrate, solubility of the substrate in water, presence of auxiliary ingredients (e.g. essential oils, clouding agents, vitamins, etc.), compatibility of SAIB and substrate, and numerous others.

In beverage applications, it is preferred that the substrate weight percent be minimized in order to minimize the amount of substrate added to the beverage. This is especially important in beverage applications, if the substrate is not soluble in water, such as waxes, silica, titanium dioxide, microcrystalline cellulose and the like. Such ingredients are not often used in beverages in an amount greater than about one percent by weight; however it is possible to disperse them in water, so that they will resist the tendency to settle out of the final beverage. In beverage applications, for substrates insoluble in water, the preferred SAIB weight percent is about 50% to about 90%, most preferably about 80 to about 90%.

Substrates that are soluble in water are preferred for beverage applications. The most preferred substrates are those that are commonly used in beverage manufacturing, including modified food starch, gum acacia and sucrose. The preferred SAIB weight percent is about 40% to about 60%, and the most preferred weight percent is about 40% to about 55%. Substrates that are insoluble in water may be preferred for cosmetic applications. If an insoluble substrate is used, the preferred range is from about 10 weight percent to about 90 weight percent, and more preferably from about 20 weight percent to about 80 weight percent.

The present invention is further related to a process for preparing a solid sucrose acetate isobutyrate formulation that is pourable in less than 20 seconds according to ASTM method D1895-96 comprising combining a sucrose acetate butyrate and a substrate.

SAIB and substrate can be combined by any suitable means known in the art, such as direct mixing, extrusion coating, spray drying, blending, and encapsulation.

SAIB and a substrate may be combined using a spray drying process. In the spray drying process the formulation are generally prepared by a three step operation comprising: (1) forming an emulsion of the SAIB, substrate, and any optional auxiliary processing aid in an aqueous solution; (2) reducing the particles to the desired size, such as by breaking up the emulsion into droplets of desired size, e.g., in a spray nozzle, from a spinning disc, or apertured centrifugal atomizer; and (3) removing moisture in a drying environment to form the SAIB formulation. The drying environment may be hot drying air (e.g., in a spray drying tower), a dehydrating liquid (e.g., propylene glycol); a bed of dehydrating powder (e.g., dry starch powder); or the like. The formulations produced by this process vary significantly depending upon the type of substrate used. While the SAIB formulation produced by the spray drying may be of various sizes and shapes and may be hollow or be substantially uniform throughout, the formulation is characterized by cellular structure comprising many dispersed globules of the core material in a matrix of the coating material. The formulation produced by the spray drying process is a dry, somewhat porous powder consisting of roughly aspherical, convoluted particles with the coating material in the solid state and with the SAIB either dispersed as minute droplets throughout the particle, or dissolved in a solid matrix, or both, depending on the compatibility of the SAIB and the substrate.

Combining the SAIB and the substrate can be accomplished in any number of other ways known in the art of mixing liquids and/or solids, such as direct mixing. These include Henschel mixer, Lodige mixer, and V-mixer, and mixing methods based principally on a shear effect such as a colloid mill, ball mill, motorized orbiting mortar and pestle, and roll mill.

Combining the SAIB and the substrate can also be accomplished using a single- or twin-screw extruder. Generally speaking, extruders are industrial devices which include an elongated, tubular barrel, a material inlet at one end of the barrel and a restricted orifice die adjacent the remaining end thereof. One or more elongated, axially rotatable, flighted extrusion screws are situated within the barrel, and serve to transport material along the length thereof. Moreover, the overall extruder is designed to heat, pressurize and render flowable material being processed, typically through the use of high shear and temperature conditions.

An example of an extruder that may be used to combine SAIB and a substrate is the single screw extruder, which includes a single, elongated extruder screw within a substantially circular barrel. Another example of extruders is the so-called twin-screw machines, which have a pair of juxtaposed elongated, flighted screws within a complemental barrel having a pair of side-by-side, frusto-cylindrical sections. The screws in such a twin screw machine can be counter rotating (i.e., the screws rotate in an opposite direction relative to each other), or co-rotating, (i.e. both screws rotate either clockwise or counterclockwise).

Such a process would have two streams commingled at the opening of the extruder: one would be a stream of SAIB, heated to 50-80° C. and the second stream would be a stream of substrate preferably in powder form. The final formulation exits the extruder in the form of a coarse powder like material or “chopped spaghetti” like material depending upon the conditions under which the extruder is operated.

In one embodiment of the present invention, the SAIB formulation can include additional components, such as processing aids useful for facilitating the combination of SAIB and the substrate composition, surfactants, diluent solvents, or other components depending on the application, such as triglycerides in beverage applications.

Processing aids may or may not be present in the final SAIB formulation. For example if spray drying is used to prepare the SAIB formulation, then the processing aids might include an organic solvent to help facilitate aqueous emulsification of the SAIB prior to spray drying. Such organic solvents include, but are not limited to, ethanol, acetone, medium chain triglycerides, ethyl acetate, and the like.

In addition, the use of emulsifiers may be used to facilitate emulsification. In a preferred embodiment, the emulsifiers are those commonly used in food, beverage, cosmetic, and pharmaceutical applications, including but not limited to: mono and di-fatty acid esters of glycerin, mono-, di-, and tri-esters of sucrose, sorbitan esters, polysorbates, steroyl lactylates, and lecithin derivatives. The amount of the emulsifier used will depend on the application.

Direct combination of SAIB and substrate may involve the use of a dilution solvent to reduce the viscosity of the SAIB to facilitate direct combination of the SAIB and the substrate. Such solvents can be subsequently removed by drying to an acceptable residual level, such as <100 ppm.

Depending on the application, many different additives can be included in the SAIB formulation. Specifically, in beverage applications medium chain triglycerides may be added to the SAIB formulation. In cosmetic applications. In cosmetic applications additives may include plasticers, oils, plant extracts, film-forming polymers, pigments, aromas, inorganic salts, water, solvents of various types, waxes, and on and on.

In beverage applications, the particle size of the water-insoluble components of the beverage emulsion is preferably reduced employing any suitable apparatus known in the art. Because the ability of emulsifying agents to hold oil in suspension is proportional to particle size, emulsions of particles with diameters of about 0.1 to about 3.0 microns are suitable for use in this invention. Preferably, the particles are about 2.0 microns or less in diameter. Most preferred is an emulsion in which substantially all the particles are 1.0 microns to about 0.4 microns in diameter. The particle size is reduced by passing the mixture through a homogenizer, colloid mill or turbine-type agitator. Usually one or two passes is sufficient.

This invention can be further illustrated by the following examples of preferred embodiments thereof, although it will be understood that these examples are included merely for purposes of illustration and are not intended to limit the scope of the invention unless otherwise specifically indicated.

In the following examples the test for pourability is ASTM method D1895-96 entitled “Apparent Density, Bulk Factor, and Pourability of Plastic Materials” which describes a pourability procedure which uses a funnel and a measured weight of sample which is timed as it flows from the funnel. The funnel described in ASTM D1895-96, “Pourability”, 20 Apparatus, page 452, was not available. A plastic vitri 964/10 funnel, having the following dimensions was used:

| | |
|---|---|
| Bottom opening | 2.3 cm |
| Bottom length before angling (spout length) | 2.5 cm |
| Top opening | 10.0 cm |
| Total height (top to bottom) | 9.5 cm |

Samples of SAIB and the SAIB formulation (samples) were poured out on a piece of paper and any clumps present were dispersed with a spatula. 10.02±0.1 grams of samples were then weighed into a glass beaker and poured into the funnel (described above). The bottom part of the funnel was blocked with the glass bottom of a small glass beaker. The beaker was removed and the time determine for the entire sample to flow through the funnel. As used herein the term “pourable” or “pourability” means that the formulation can be poured according to ASTM method D1895-96 in less than 20 seconds.

### EXAMPLE 1a

### Preparation of SAIB/Starch Formulation

An aqueous solution of modified food starch was prepared from 143 g of EmCap 12633 (commercially available form Cargill, Inc., Hammond, Ind.) and 574 g of demineralized water. To this was added under high shear using a Gifford-Wood homogenizer, a solution consisting of 50 g sucrose acetate isobutyrate (SAIB, commercially available for Eastman Chemical Company, Kingsport, Tenn.) and 45 g of ethanol. The resulting emulsion was then spray dried using an APV Anhydro Model Lab 1 spray dryer. The spray dryer operating conditions were:

| | |
|---|---|
| Inlet temperature | 75° C. |
| Outlet temperature | 55° C. |
| Atomization pressure | 35 psig |
| Spray rate | 34.6 g/minute |
| Product form | <5 seconds |

### EXAMPLE 1b

### Preparation of SAIB/Starch/Medium Chain Triglycerides Formulation

An aqueous solution of modified food starch was prepared from 143 g of EmCap 12633 (commercially available form Cargill, Inc., Hammond, Ind.) and 574 g of demineralized water. To this was added under high shear using a Gifford-Wood homogenizer, a solution consisting of 100 g sucrose acetate isobutyrate (SAIB, commercially available for Eastman Chemical Company, Kingsport, Tenn.), 10 g of ethanol, and 20 g medium chain triglycerides (commercially known as Neobee M5 and available from Stepan Company, Northfield, Ill.). The resulting emulsion was then spray dried using an APV Anhydro Model Lab 1 spray dryer. The spray dryer operating conditions were the same as above. The resulting formulation was pourable in less than 5 seconds and contained approximately 18-22% medium chain triglycerides of the final formulation.

### EXAMPLE 2

### Alternate Procedure for Making SAIB/Starch Formulation

In a 1L round-bottomed flask was combined 50 grams of modified food starch (Purity Gum 1773 commercially available from National Starch and Chemical, Bridgewater, N.J.) and a solution prepared from 50 grams of sucrose acetate isobutyrate and 100 ml of ethanol. The mixture was evaporated to dryness using a rotary evaporator operated with a vacuum of 10 mm Hg vacuum and water bath temperature of approximately 50° C. The resulting formulation was pourable (less than 5 seconds).

### EXAMPLE 3a

### Preparation of SAIB/Acacia Gum Formulation

An aqueous solution of acacia gum was prepared from 100 g of Instant Gum AS IRX 40830 (commercially available form Colloides Naturels International, Rouen Cédex, France) and 500 g of demineralized water. To this was added under high shear using a Gifford-Wood homogenizer, a solution consisting of 100 g sucrose acetate isobutyrate (SAIB, commercially available for Eastman Chemical Company, Kingsport, Tenn.) and 10 g of ethanol. The resulting emulsion was then spray dried using an APV Anhydro Model Lab 1 spray dryer to form an SAIB formulation. The spray dryer operating conditions were:

| | |
|---|---|
| Inlet temperature | 83° C. |
| Outlet temperature | 60° C. |
| Atomization pressure | 35 psig |
| Spray rate | 32 g/min |
| Product form | <5 seconds |

### EXAMPLE 3b

### Preparation of SAIB/Acacia Gum/Medium Chain Triglycerides Formulation

An aqueous solution of acacia gum was prepared from 100 g of Instant Gum AS IRX 40830 (commercially available form Colloides Naturels International, Rouen Cédex, France) and 500 g of demineralized water. To this was added under high shear using a Gifford-Wood homogenizer, a solution consisting of 100 g sucrose acetate isobutyrate (SAIB, commercially available for Eastman Chemical Company, Kingsport, Tenn.), 10 g of ethanol, and 20 g medium chain triglycerides (commercially known as Neobee M5 and available from Stepan Company, Northfield, Ill.) The resulting emulsion was then spray dried using an APV Anhydro Model Lab 1 spray dryer. The spray dryer operating conditions were the same as above. The resulting formulation was pourable (less than 5 seconds) and contained approximately 18-22% medium chain triglycerides by weight of the final formulation.

### EXAMPLE 4

### Preparation of SAIB/Maltodextrin Fiber Formulation

An aqueous solution of maltodextrin powder was prepared from 143 g of Fibersol-2 (commercially available form Matsutani America, Inc., Decatur, Ill.) and 574 g of demineralized water. To this was added under high shear using a Gifford-Wood homogenizer, a solution consisting of 45 g sucrose acetate isobutyrate, 45 g of ethanol, and 5 g of Neobee M5 (medium chain triglyceride blend, commercially available from Stepan Company, Northfield, Ill.). The resulting emulsion was then spray dried using an APV Anhydro Model Lab 1 spray dryer to form an SAIB formulation. The spray dryer operating conditions were:

| | |
|---|---|
| Inlet temperature | 80° C. |
| Outlet temperature | 60° C. |
| Atomization pressure | 35 psig |
| Spray rate | 34.9 g/minute |
| Yield | 80 g (not optimized) |
| Product form | <5 seconds |

### EXAMPLE 5

### Preparation of SAIB/Sucrose Formulation

An aqueous solution of sucrose was prepared from 500 g of sucrose and 2000 g of demineralized water. To this was added under high shear using a Gifford-Wood homogenizer, a solution consisting of 500 g sucrose acetate isobutyrate, 200 g of ethanol, and 1 g of sodium dioctylsulfosuccinate surfactant, commercially available from Cytec Industries, West Patterson, N.J.). The resulting emulsion was then spray dried using an APV Anhydro Model Lab 1 spray dryer to form an SAIB formulation. The spray dryer operating conditions were:

| | |
|---|---|
| Inlet temperature | 74° C. |
| Outlet temperature | 52° C. |
| Atomization pressure | 35 psig |
| Spray rate | 23.7 g/minute |
| Yield | 20 g (not optimized) |
| Product form | <5 seconds |

### EXAMPLE 6

### Preparation of SAIB/Silicon Dioxide Formulation

In a 1 L round-bottomed flask were combined 45 grams of silicon dioxide (available commercially as Zeosyl 200 from J. M. Huber Corporation, Havre de Grace, Md.) and a solution of 30 grams of sucrose acetate isobutyrate dissolved in 100 ml of ethanol. The mixture was evaporated to dryness using a rotary evaporator operated with 10 mm Hg vacuum and water bath temperature of approximately 50° C. The resulting powder was free-flowing.

### EXAMPLE 7

### Preparation of SAIB/Bees Wax Formulation

Bees wax (from Aldrich Chemical Company, Milwaukee, Wis.)), 100 g, was melted at 60-70° C. in a beaker. Sucrose acetate isobutyrate was added in a single shot and the mixture was stirred by a mechanical agitator as the temperature was allowed to drift downward until the wax hardened. At 25° C., the wax/sucrose acetate isobutyrate mixture was hard and non-tacky. It was readily millable to form a coarse, pourable SAIB formulation.

### EXAMPLE 8

### Preparation of SAIB/Candellila Wax Formulation

Candellila wax (from Aldrich Chemical Company, Milwaukee, Wis.), 100 g, was melted at 60-70° C. in a beaker. Sucrose acetate isobutyrate was added in a single shot and the mixture was stirred by a mechanical agitator as the temperature was allowed to drift downward until the wax hardened. At 25° C., the wax/sucrose acetate isobutyrate mixture was hard and non-tacky. It was readily millable to form a coarse, pourable SAIB formulation.

### EXAMPLE 9

### Preparation of SAIB/Hexadecyl Hexadecanoate Wax Formulation

Hexadecyl hexadecanoate (from Aldrich Chemical Company, Milwaukee, Wis.), 100 g, was melted at 70-80° C. in a beaker. Sucrose acetate isobutyrate was added in a single shot and the mixture was stirred by a mechanical agitator as the temperature was allowed to drift downward until the wax hardened. At 25° C., the wax/sucrose acetate isobutyrate mixture was hard and non-tacky. It was readily millable to form a coarse, pourable (less than 5 seconds) formulation.

### EXAMPLE 10

### Preparation of SAIB/Hydrocarbon Wax Formulation

Hydrocarbon wax (known as CRW 141 and commercially available from Chevron Products Company, San Ramon, Calif.), 100 g, was melted at 60-70° C. in a beaker. Sucrose acetate isobutyrate was added in a single shot and the mixture was stirred by a mechanical agitator as the temperature was allowed to drift downward until the wax hardened. At 25° C., the wax/sucrose acetate isobutyrate mixture was hard and non-tacky. It was readily millable to form a coarse, pourable SAIB formulation.

### EXAMPLE 11

### Preparation of Beverage Emulsion Using SAIB/Starch Formulation

A blend of four parts single-fold orange and one part 5-fold orange oil was prepared for use as the flavoring oil. The oil phase was prepared by combining 32.7 grams of the above orange oil blend and 100.1 grams of SAIB/starch formulation prepared according to the recipe of Example la. The oil phase slurry containing starch powder was stirred mechanically for approximately 15 minutes, then combined with an aqueous phase containing 973.5 grams of water, 137.4 g of modified food starch (EmCap 12633, commercially available form Cargill, Inc., Hammond, Ind.), 4.4 g of citric acid, and 1.9 g of sodium benzoate and then homogenized using a GreerCo Gifford-Wood High Shear Mixer. The resulting emulsion was de-aerated for 18 hours and then homogenized at 6000 psi (two passes) using a two-staged homogenizer (Model 15MR-8TA from APV Gaulin, Inc.). The particle size distribution was determined using a Microtrac UPA instrument. Approximately 99.5% of the oil droplets measured less than 1.06 microns, with Mv=0.53 and Mn=0.33, where Mv is the mean diameter of the volume distribution, and Mn is the mean diameter of the number distribution. Mv is influenced strongly by the number or coarse particles present, while Mn is weighted to small particles.

Percent Composition of Emulsion

| | |
|---|---|
| Orange oil | 2.6% |
| SAIB | 4.0% |
| Starch emulsifier | 15.0% |
| Sodium benzoate, preservative | 0.1% |
| Citric acid, acidulate | 0.35% |
| Water | 78.0% |
| Total | 100% |

The calculated specific gravity of the oil phase was 1.008

### EXAMPLE 12

### Preparation of Beverage Syrup from Emulsion of Example 11

A beverage syrup was prepared by combining 3 grams of emulsion prepared in Example 10 with an aqueous sugar solution containing 105.6 g sucrose, 0.3 g sodium benzoate, 1.3 g citric acid, and 84.8 grams of water.

### EXAMPLE 13

### Preparation of Carbonated Beverage from Syrup of Example 12

A carbonated beverage was prepared by combining in a plastic beverage bottle 80 g of syrup prepared in Example 11 with 400 g of water saturated with carbon dioxide. The turbidity of the final beverage was measured using a Hach turbidimeter Model Ratio/XR. A water blank was used. The container was sealed and shelved for observation. The beverage emulsion continued to be homogeneous with no signs of layer separation or lifting. The final beverage and beverage syrup remained cloudy without formation of any sediment or oil separation. The turbidity of the freshly prepared beverage was determined to be 227 NTU.

### EXAMPLE 14

### Preparation of Beverage Emulsion Using SAIB/Acacia Formulation

A blend of four parts single-fold orange and one part 5-fold orange oil was prepared for use as the flavoring oil. The oil phase was prepared by combining 32.8 grams of the above orange oil blend and 100 grams of SAIB/acacia powder prepared according to the recipe of Example 3a. The oil phase slurry containing acacia powder was stirred mechanically for approximately 15 minutes, then combined with an aqueous phase containing 973.5 grams of water, 137 g of acacia gum (commercially available form Colloides Naturels International, Rouen Cédex, France), 4.4 g of citric acid, and 1.9 g of sodium benzoate and then homogenized using a GreerCo Gifford-Wood High Shear Mixer. The resulting emulsion was de-aerated for 18 hours and then homogenized at 6000 psi (two passes) using a two-staged homogenizer (Model 15MR-8TA from APV Gaulin, Inc.). The particle size distribution was determined using a Microtrac UPA instrument. Approximately 99.9% of the oil droplets measured less than 1.06 microns, with Mv=0.63 and Mn=0.50, where Mv is the mean diameter of the volume distribution, and Mn is the mean diameter of the number distribution. Mv is influenced strongly by he number or coarse particles present, while Mn is weighted to small particles.

Percent Composition of Emulsion

| | |
|---|---|
| Orange oil | 2.6% |
| SAIB | 4.0% |
| Acacia gum emulsifier | 15.0% |
| Sodium benzoate, preservative | 0.1% |
| Citric acid, acidulate | 0.35% |
| Water | 78.0% |
| Total | 100% |

The calculated specific gravity of the oil phase was 1.008

### EXAMPLE 15

### Preparation of Beverage Syrup from Emulsion of Example 14

A beverage syrup was prepared by combining 3 grams of emulsion prepared in Example 14 with an aqueous sugar solution containing 105.6 g sucrose, 0.3 g sodium benzoate, 1.3 g citric acid, and 84.8 grams of water.

### EXAMPLE 16

### Preparation of Carbonated Beverage from Syrup of Example 15

A carbonated beverage was prepared by combining in a plastic beverage bottle 80 g of syrup prepared in Example 14 with 400 g of water saturated with carbon dioxide. The turbidity of the final beverage was measured using a Hach turbidimeter Model Ratio/XR. A water blank was used. The container was sealed and shelved for observation. The beverage emulsion continued to be homogeneous with no signs of layer separation or lifting. The final beverage and beverage syrup remained cloudy without formation of any sediment or oil separation. The turbidity of the freshly prepared beverage was determined to be 161 NTU.

### EXAMPLE 17

### Preparation of Beverage Emulsion Using SAIB/Acacia Powder/Medium Chain Triglycerides

A blend of four parts single-fold orange and one part 5-fold orange oil was prepared for use as the flavoring oil. The oil phase was prepared by combining 32.8 grams of the above orange oil blend and 94 grams of SAIB/acacia gum/medium chain triglycerides (approx. 20% medium chain triglyceride content) powder prepared according to the recipe of Example 3b. The oil phase slurry containing acacia powder was stirred mechanically for approximately 15 minutes, then combined with an aqueous phase containing 973.5 grams of water, 137 g of acacia gum (commercially available form Colloides Naturels International, Rouen Cédex, France), 4.4 g of citric acid, and 1.9 g of sodium benzoate and then homogenized using a GreerCo Gifford-Wood High Shear Mixer. The resulting emulsion was de-aerated for 18 hours and then homogenized at 6000 psi (two passes) using a two-staged homogenizer (Model 15MR-8TA from APV Gaulin, Inc.). The particle size distribution was determined using a Microtrac UPA instrument. Approximately 97.9% of the oil droplets measured less than 1.06 microns, with Mv=0.67and Mn=0.56, where Mv is the mean diameter of the volume distribution, and Mn is the mean diameter of the number distribution. Mv is influenced strongly by he number or coarse particles present, while Mn is weighted to small particles.

Percent Composition of Emulsion

| | |
|---|---|
| Orange oil | 2.6% |
| SAIB | 3.1% |
| Acacia gum emulsifier | 15.0% |
| Sodium benzoate, preservative | 0.1% |
| Citric acid, acidulate | 0.35% |
| Water | 78.8% |
| Total | 100% |

The calculated specific gravity of the oil phase was 1.008

### EXAMPLE 18

### Preparation of Beverage Syrup from Emulsion of Example 17

A beverage syrup was prepared by combining 3 grams of emulsion prepared in Example 17 with an aqueous sugar solution containing 105.6 g sucrose, 0.3 g sodium benzoate, 1.3 g citric acid, and 84.8 grams of water.

### EXAMPLE 19

### Preparation of Carbonated Beverage from Syrup of Example 18

A carbonated beverage was prepared by combining in a plastic beverage bottle 80 g of syrup prepared in Example 17 with 400 g of water saturated with carbon dioxide. The turbidity of the final beverage was measured using a Hach turbidimeter Model Ratio/XR. A water blank was used. The container was sealed and shelved for observation. The beverage emulsion continued to be homogeneous with no signs of layer separation or lifting. The final beverage and beverage syrup remained cloudy without formation of any sediment or oil separation. The turbidity of the freshly prepared beverage was determined to be 277 NTU.

The invention has been described in detail with particular reference to preferred embodiments thereof, but it will be understood that variations and modifications can be effected within the spirit and scope of the invention.

1. A formulation comprising:
sucrose acetate isobutyrate in an amount from about 1 weight percent to about 80 weight percent based on the weight percent of the formulation; and
a substrate, wherein the substrate is present in an amount from about 30 weight percent to about 99 weight percent based on the weight percent of the formulation,
wherein the formulation is pourable in less than about 20 seconds according to ASTM method D1895-96.

2. A formulation as recited in claim 1, wherein the sucrose acetate isobutyrate is present in an amount from about 30 weight percent to about 70 weight percent.

3. A formulation as recited in claim 1, wherein the sucrose acetate isobutyrate is present in an amount from about 40 weight percent to about 60 weight percent.

4. A formulation as recited in claim 1, wherein the sucrose acetate isobutyrate is present in an amount from about 45 weight percent to about 55 weight percent.

5. A formulation as recited in claim 1, wherein the substrate is present in an amount from about 40 weight percent to about 60 weight percent.

6. A formulation as recited in claim 1, wherein the formulation is pourable in less than about 10 seconds.

7. A formulation as recited in claim 1, wherein the formulation is pourable in less than about 5 seconds.
